(19) United States
(12) Patent Application Publication
(10) Pub. No.: US 2025/0082503 A1
(43) Pub. Date: Mar. 13, 2025

(54) EXTERNAL FIBER FOR OPTICAL FIBER DELIVERY IN OPTHALMIC SURGICAL DEVICES

(71) Applicant: Alcon Inc., Fribourg (CH)

(72) Inventors: Kevin Canzone, Coto de Caza, CA (US); John Morgan Bourne, Irvine, CA (US); Mikhail Ovchinnikov, Dana Point, CA (US)

(21) Appl. No.: 18/819,041

(22) Filed: Aug. 29, 2024

Related U.S. Application Data
(60) Provisional application No. 63/582,009, filed on Sep. 12, 2023.

(51) Int. Cl. A61F 9/008

(52) U.S. Cl. CPC A61F 9/0084; A61F 9/00814; A61B 2218/002; A61B 2218/007; A61F 2009/0087; A61F 2009/00874

(57) ABSTRACT

Embodiments disclosed herein provide an optical fiber system. The optical fiber system includes an aspiration tube, an optical fiber external to the aspiration tube, and a sleeve surrounding the optical fiber and the aspiration tube. The sleeve includes a distal portion that defines a channel. The channel extends between the aspiration tube and a distal opening of the sleeve. The channel and the aspiration tube define an aspiration pathway. The optical fiber is partially disposed within the channel.

### BACKGROUND

In a wide variety of medical procedures, laser light is used to perform surgery and/or to treat patient anatomy. For example, in laser phacoemulsification, a laser probe propagates a laser beam to emulsify and ablate the crystalline lens for cataract removal. A laser beam is typically transmitted from a surgical laser system through an optical fiber that proximally terminates in a port adaptor connected to the surgical laser system, and distally terminates in the laser probe, which is manipulated by a surgeon.

In some laser probes, the optical fibers are housed within an aspiration tube. The housing of the optical fiber within the aspiration tube can cause issues with clogging, as the optical fiber occupies a portion of the aspiration pathway. The optical fiber may further block the aspiration pathway by not allowing larger pieces of tissue and vitreous to be aspirated out of the aspiration tube. As a result, the laser probe may become less ineffective in removing the tissue and vitreous produced during the medical procedure.

### SUMMARY

The present disclosure relates generally to optical fibers, and more specifically, to components for energy delivery in surgical systems.

Certain embodiments of the present disclosure provide an optical fiber system. The optical fiber system includes an aspiration tube, an optical fiber external to the aspiration tube, and a sleeve surrounding the optical fiber and the aspiration tube. The sleeve includes a distal portion that defines a channel. The channel extends between the aspiration tube and a distal opening of the sleeve. The channel and the aspiration tube define an aspiration pathway. The optical fiber is partially disposed within the channel.

Certain embodiments of the present disclosure provide an optical fiber system. The optical fiber system includes an aspiration tube, an optical fiber external to the aspiration tube, and a distal cap. The distal cap includes a channel extending between the aspiration tube and a distal opening. The channel and the aspiration tube define an aspiration pathway. The optical fiber is partially disposed within the channel.

Certain embodiments of the present disclosure provide an optical fiber system. The optical fiber system includes an aspiration tube, an optical fiber external to the aspiration tube, and a distal cap. The distal cap includes a hypotube disposed at a distal end of the aspiration tube and a channel. The hypotube includes a hypotube opening. The channel extends between the aspiration tube and the distal opening. The channel and the aspiration tube define an aspiration pathway. The hypotube is disposed within the channel.

The following description and the related drawings set forth in detail certain illustrative features of one or more embodiments, including those described above.

### BRIEF DESCRIPTION OF THE DRAWINGS

So that the manner in which the above-recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, and may admit to other equally effective embodiments.

FIG. 1 illustrates a side view of a system for generating laser beams for delivery to a surgical target, in accordance with certain embodiments of the present disclosure.

FIG. 2A illustrates a perspective view of a probe tip of a probe having a sleeve, in accordance with certain embodiments of the present disclosure.

FIG. 2B illustrates a cross-sectional side view of the distal portion of the probe of FIG. 2A, in accordance with certain embodiments of the present disclosure.

FIG. 2C illustrates a cross-sectional side view of a distal portion the sleeve of FIG. 2A, in accordance with certain embodiments of the present disclosure.

FIG. 2D illustrates a perspective view of the distal portion of the sleeve of FIG. 2A, in accordance with certain embodiments of the present disclosure.

FIG. 3A illustrates a perspective view of a probe tip of a probe having a distal cap, in accordance with certain embodiments of the present disclosure.

FIG. 3B illustrates a cross-sectional side view of the probe tip of the probe of FIG. 3A, in accordance with certain embodiments of the present disclosure.

FIG. 3C illustrates a perspective view of the distal cap of FIG. 3A, in accordance with certain embodiments of the present disclosure.

FIG. 3D illustrates a cross-sectional side view of the distal cap of FIG. 3A, in accordance with certain embodiments of the present disclosure.

FIG. 3E illustrates a cross-sectional side view of an optical fiber and an aspiration tube without the distal cap of FIG. 3A, in accordance with certain embodiments of the present disclosure.

FIG. 3F illustrates a perspective view of the optical fiber and the aspiration tube of FIG. 3E without the distal cap of FIG. 3A, in accordance with certain embodiments of the present disclosure.

FIG. 4A illustrates a cross-sectional side view of the distal cap of FIG. 3A including a hypotube, in accordance with certain embodiments of the present disclosure.

FIG. 4B illustrates a cross-sectional side view of an optical fiber and hypotube of FIG. 4A without the distal cap of FIG. 3A, in accordance with certain embodiments of the present disclosure.

FIG. 4C illustrates a perspective view of the optical fiber and the hypotube of FIG. 4A without the distal cap of FIG. 3A, in accordance with certain embodiments of the present disclosure.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

In the following description, details are set forth by way of example to facilitate an understanding of the disclosed subject matter. It should be apparent to a person of ordinary skill in the field, however, that the disclosed implementations are exemplary and not exhaustive of all possible implementations. Thus, it should be understood that reference to the described examples is not intended to limit the scope of the disclosure. Any alterations and further modifications to the described devices, instruments, methods, and any further application of the principles of the present disclosure are fully contemplated as would normally occur to one skilled in the art to which the disclosure relates. In particular, it is fully contemplated that the features, components, and/or steps described with respect to one implementation may be combined with the features, components, and/or steps described with respect to other implementations of the present disclosure.

Note that, as described herein, a distal end, segment, or portion of a component refers to the end, segment, or portion that is closer to a patient's body during use thereof. On the other hand, a proximal end, segment, or portion of the component refers to the end, segment, or portion that is distanced further away from the patient's body and is in proximity to, for example, a surgical laser system.

As used herein, the term “about” may refer to a +/−10% variation from the nominal value. It is to be understood that such a variation can be included in any value provided herein.

Particular embodiments disclosed herein generally relate to surgical systems, and more specifically, to components for energy delivery in surgical systems.

During surgery and/or treating patient anatomy, optical fibers of various types may have transmission properties that allow the optical fibers to efficiently and accurately guide a laser beam from a laser source to a surgical site. However, as discussed, housing an optical fiber within an aspiration tube of a laser probe can cause issues with clogging, since the optical fiber occupies a portion of the aspiration pathway. The optical fiber may further block the aspiration pathway by not allowing larger pieces of tissue and vitreous to be aspirated out of the aspiration tube. As a result, the laser probe may become less ineffective in removing the tissue and vitreous produced during the medical procedure.

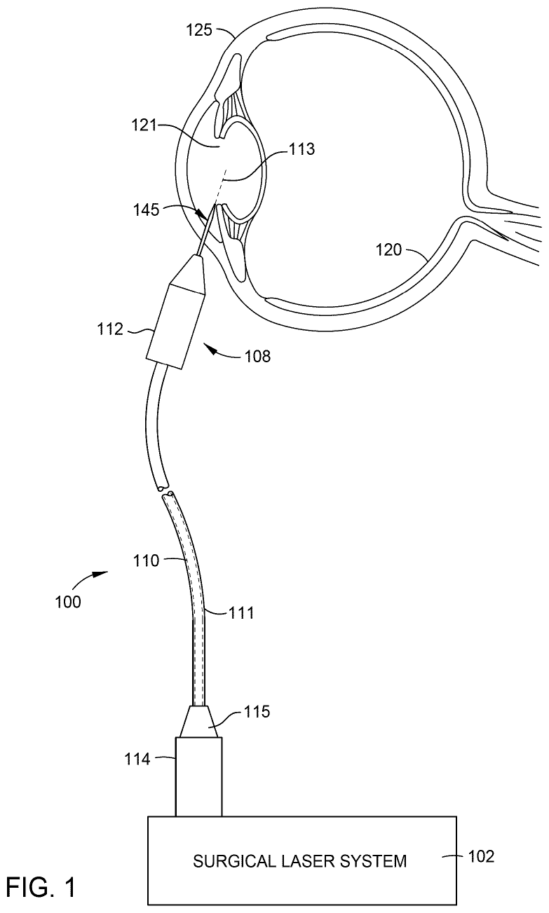

Aspects of the present disclosure provide for surgical systems for more effectively removing tissue and vitreous produced during a medical procedure. The surgical system may include an optical fiber positioned outside of the aspiration pathway of a laser probe that is used to emulsify and aspirate lens material. By positioning the optical fiber outside of the aspiration pathway, the risk of clogging can be significantly reduced. The optical fiber may be positioned adjacent to the aspiration tube and partially disposed within a channel at a distal end of the probe. In various embodiments described herein, the channel may be defined by either a sleeve or a distal cap which positions the optical fiber to efficiently deliver the laser beams to the surgical site. Such embodiments are described below in further detail in conjunction with FIGS. 1, 2A-2D, 3A-3F, and 4A-4C.

FIG. 1 illustrates an example surgical system 100 for performing a laser-assisted ophthalmic procedure. Surgical system 100 includes a laser system 102, having one or more laser sources for generating a laser beam 113. In one example, the laser beam 113 may have a wavelength of about 1 μm (micrometer) to about 10 μm, such as about 3 μm and may be suitable for phacoemulsification. The user, such as a surgeon, may toggle the laser between on positions and off positions using a switch on the probe 108, a foot pedal, or other means.

The surgical system 100 includes a connector (e.g., port adaptor 114), an optical fiber 110, an optical fiber cable 111, and a probe 108. The optical fiber 110 may be at least partially housed inside the optical fiber cable 111. A distal end of the optical fiber cable 111 couples to the probe 108, and a proximal end of the optical fiber cable 111 couples to a port adaptor 114. In some cases, the optical fiber 110 may include more than one fiber. The optical fiber 110 may have a transmissibility in the range of about 1 μm to about 10 μm, such as about the 3 μm wavelength range.

The port adaptor 114 couples to an optical port of laser system 102. The optical fiber 110 extends through the port adaptor 114 toward the optical port. The port adaptor 114 may include a ferrule 115 with an opening into which a proximal end of optical fiber 110 and a proximal end of the optical fiber cable 111 are inserted.

The probe 108 includes a probe body 112 and a probe tip 145. The optical fiber 110 extends through the probe body 112 to a distal end portion of the probe tip 145. Optical fiber 110 delivers laser beams through probe 108, which guides the laser beams to a surgical site (e.g., a crystalline lens 121) of a patient's eye 125. The probe body 112 and the probe tip 145 house and protect the distal end of optical fiber 110.

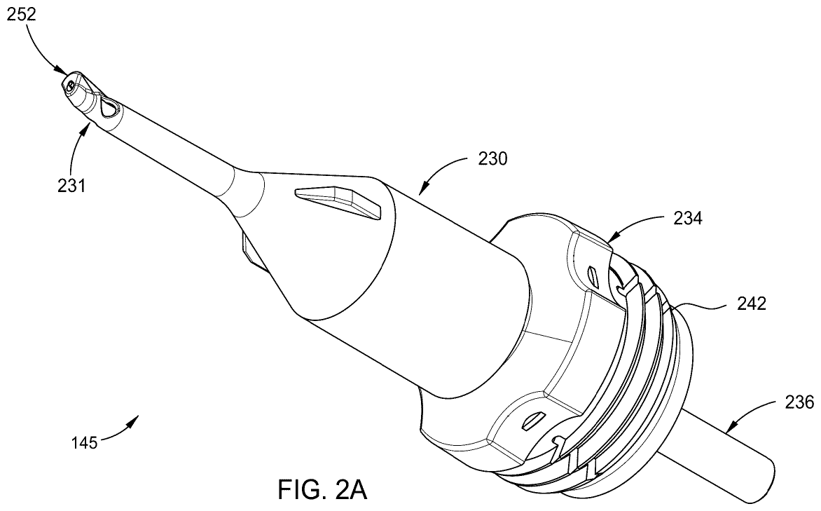

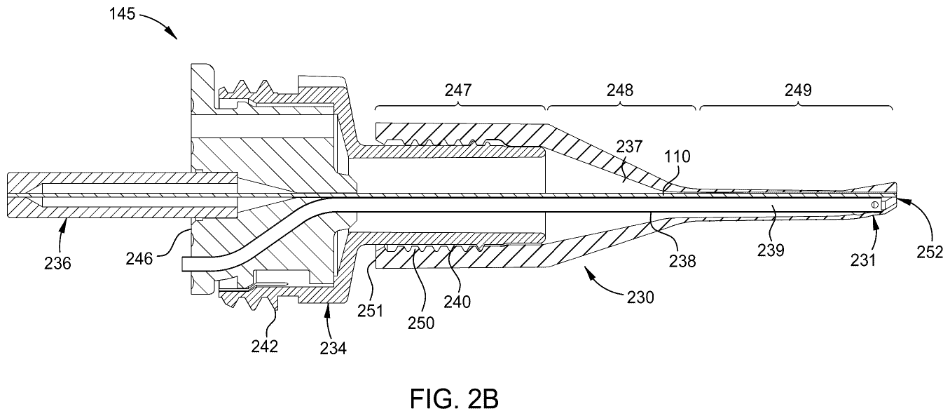

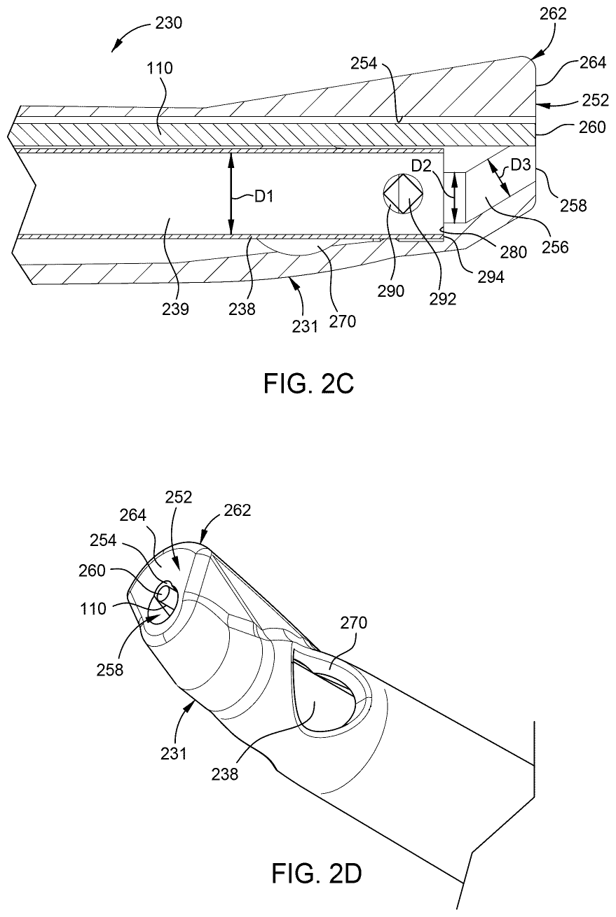

FIG. 2A-2D illustrate the probe tip 145 of the probe 108 having a sleeve 230. As shown in FIG. 2A and FIG. 2B, the probe tip 145 of the probe 108 includes the sleeve 230, a connection component 234, an optical fiber housing 236, a portion of the optical fiber 110, and an aspiration tube 238. The optical fiber 110 may be positioned adjacent to the aspiration tube 238, such that the optical fiber 110 is external to the aspiration tube 238. In some embodiments, the aspiration tube 238 has a cross-sectional area defined by a diameter D1. In other embodiments, the cross-sectional area may be defined by other dimensions, e.g., height and width, depending on the shape of the aspiration tube 238. By positioning the optical fiber 110 external to the aspiration tube 238, the cross-sectional area of the aspiration tube 238 through which tissue and vitreous may be aspirated is increased. Advantageously, an increase in the cross-sectional area of the aspiration tube 238 decreases the likelihood of clogging and further allows for the aspiration of larger pieces of tissue, vitreous, etc.

In various embodiments, sleeve threads 240 are included near the distal end 244 of the connection component 234, and probe threads 242 are included near the proximal end 246 of the connection component 234. The sleeve threads 240 are configured to secure the connection component 234 to a connection component threads 250 of the sleeve 230. The probe threads 242 are configured to secure the connection component 234 to the connection component threads of the probe 108. The proximal end 246 of the connection component 234 is coupled to the optical fiber housing 236. The optical fiber housing 236 houses a portion of the optical fiber 110.

As shown in FIG. 2B, the sleeve 230 may include a proximal region 247, an intermediate region 248, and a distal region 249. The sleeve 230 may include a silicone material, an elastomeric material, a plastic material, a metal material, or a ceramic material. The connection component threads 250 are near the proximal end 251 of the sleeve 230 within the proximal region 247. In some embodiments, the connection component 234 is secured to the sleeve 230 by threading the connection component threads 250 onto the sleeve threads 240. In other embodiments, the connection component 234 is secured to the sleeve 230 by press-fitting. The intermediate region 248 connects the proximal region 247 to the distal region 249. In some embodiments, the proximal region 247 has a cross-section area (e.g., an area defined by a diameter) that is greater than a cross-sectional area (e.g., an area defined by a diameter) of the distal region 249. The intermediate region 248 may be cone-shaped, with a sidewall 237 that tapers inwardly from the proximal region 257 to the distal region 249.

As shown in FIG. 2C and FIG. 2D, the distal region 249 includes a distal portion 231, a distal end 252, and an optical fiber guide 254. The distal portion 231 of the sleeve 230 includes a channel 256 and a distal opening 258. The optical fiber guide 254 may partially surround the optical fiber 110. For example, the optical fiber guide 254 may have an annular shape that partially surrounds the optical fiber 110. The optical fiber guide 254 may terminate at the distal opening 258. In some embodiments, a distal end 260 of the optical fiber 110 is coplanar or substantially coplanar with the distal opening 258.

In some embodiments, the distal end 252 may include a distal flange 262 extending away from the distal opening 258 below the optical fiber guide 254. A distal surface 264 is formed by the distal flange 262. The distal surface 264 provides for improved followability, purchase, and to prevent burrowing of the probe tip 145. In some embodiments, the distal end 260 of the optical fiber 110 is coplanar or substantially coplanar with the distal surface 264.

The channel 256 extends between the aspiration tube 238 and the distal opening 258. The aspiration tube 238 and the channel 256 define an aspiration pathway 239. The channel 256 may include a transition region 266 and an angled region 268. The angled region 268 of the channel 256 is angled from the aspiration tube 238 towards the optical fiber guide 254 and terminates at the distal opening 258. In embodiments including the transition region 266, the angled region may be angled from the transition region 266 towards the optical fiber guide 254.

The transition region 266 may have a cross-sectional area defined by a diameter D2. In other embodiments, the cross-sectional area may be defined by other dimensions, e.g., height and width, depending on the shape of the transition region 266. The diameter D1 of the aspiration tube 238 may be greater than the diameter D2 of the transition region 266. In such embodiments, the cross-sectional area of the transition region 266 therefore limits the size of the pieces of tissue and vitreous that are capable of being aspirated through the aspiration pathway 239.

The angled region 268 may have a cross-sectional area defined by a diameter D3. In other embodiments, the cross-sectional area may be defined by other dimensions, e.g., height and width, depending on the shape of the angled region 268. The diameter D2 of the transition region 266 may be greater than or approximately equal to the diameter D2 of the angled region 268. The cross-sectional area of the angled region 268, therefore, limits the size of the pieces of tissue and vitreous that are being aspirated through the aspiration pathway 239. The distal opening 258 has a cross-sectional area substantially equal to that of a distal end of the angled region 268.

In various embodiments, the optical fiber guide 254 may position the optical fiber 110 within a portion of the channel 256 at the distal opening 258, enabling the optical fiber 110 to more efficiently deliver the laser beams to the surgical site. As a result, tissue and vitreous that is ablated and/or dissolved by the optical fiber 110 can be more effectively aspirated through the aspiration pathway 239. Accordingly, by positioning the optical fiber 110 proximate to the aspiration pathway 239, without being disposed within the aspiration tube 238, the likelihood of clogging is decreased.

Additionally, in various embodiments, the optical fiber guide 254 may position the optical fiber within a portion of the channel 256 to control the size of the tissue and vitreous which can be aspirated through the cross-sectional area of the distal opening 258. Positioning the optical fiber within a portion of the channel 256 decreases the cross-sectional area of the distal opening 258. The cross-sectional area of the distal opening 258, therefore, limits the size of the pieces of tissue and vitreous that are aspirated through the aspiration pathway 239. As a result, tissue and vitreous that is aspirated through the aspiration pathway is smaller than the cross-section area of the distal opening 258, the angled region 268, and the transition region 266, further decreasing the likelihood that large pieces of tissue and vitreous cause clogging within the aspiration pathway 239.

The sleeve 230 may further comprise one or more fluid openings 270. The fluid openings 270 are configured to allow for replacement fluids, such as silicone oil or saline, to be provided to the interior of the patient's eye as tissue and vitreous are removed. The replacement fluids prevent the loss of intraocular pressure (IOP) during the surgical procedure.

As shown in FIG. 2C, the sleeve 230 may further comprise an inner flange 280 and an anchor 292. The aspiration tube 238 may comprise an anchor opening 290. The anchor 292 may be configured to be disposed within the anchor opening 290. The inner flange 280 abuts a distal end 294 of the aspiration tube 238. As a result, the anchor 292 and inner flange 280 position the aspiration tube 238 within the sleeve 230 to allow the aspiration tube to efficiently aspirate the tissue and vitreous.

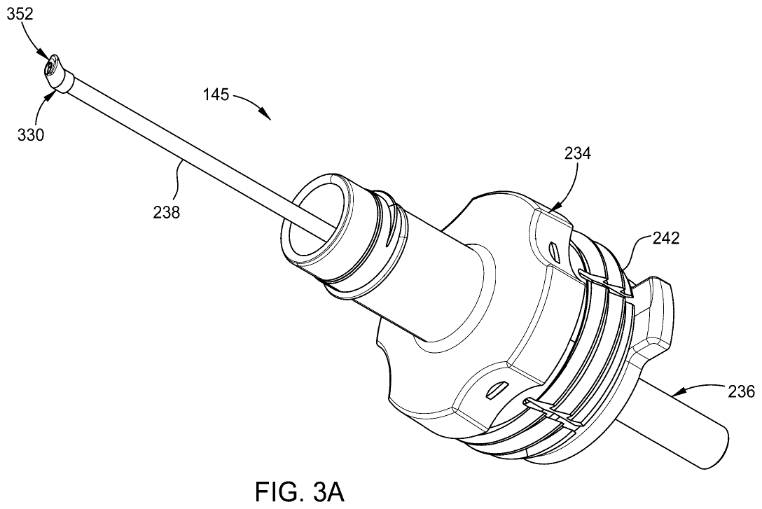

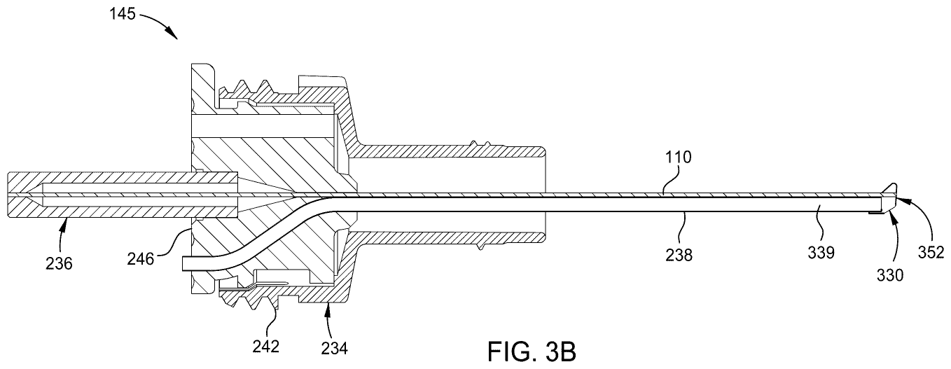

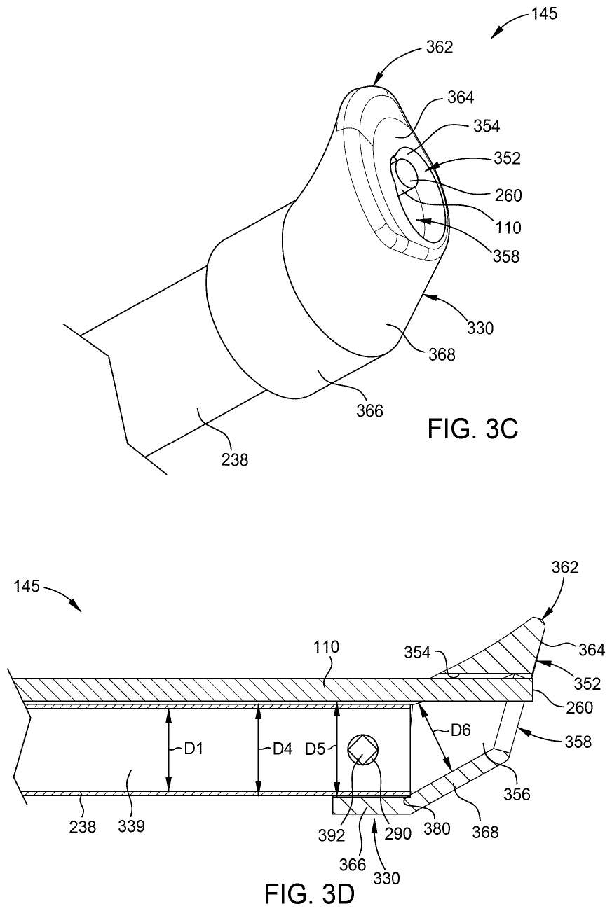

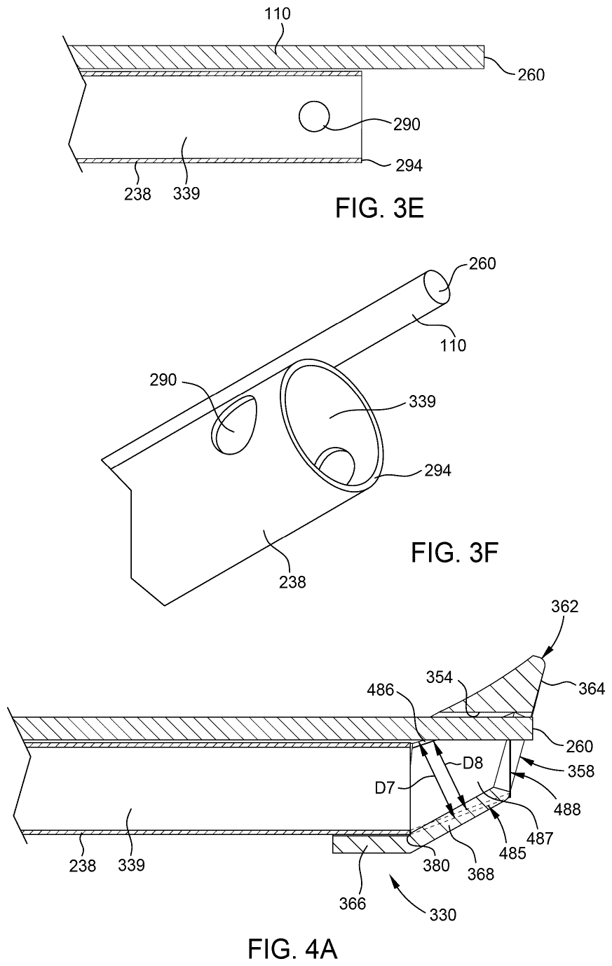

FIG. 3A-3F illustrate the probe tip 145 of the probe 108 having a distal cap 330. The probe tip 145 of the probe 108, as shown in FIG. 3A, includes the distal cap 330, a connection component 234, a portion of the optical fiber 110, and an aspiration tube 238. The aspiration tube 238 is positioned adjacent to the optical fiber 110, such that the optical fiber 110 is external to the aspiration tube 238. In some embodiments, the aspiration tube 238 has a cross-sectional area defined by a diameter D1. In other embodiments, the cross-sectional area may be defined by other dimensions (e.g., height and width), depending on the shape of the aspiration tube 238. The optical fiber 110 is external to the aspiration tube 238, increasing the cross-sectional area of the aspiration tube 238 through which tissue and vitreous may be aspirated. An increase in the cross-sectional area of the aspiration tube 238 decreases the likelihood of clogging and also allows for the aspiration of larger pieces of tissue and vitreous.

As shown in FIG. 3B, the proximal end 246 of the connection component 234 may include probe threads 242. The probe threads 242 are configured to secure the connection component 234 to the connection component threads of the probe 108. The proximal end 246 of the connection component 234 is coupled to the optical fiber housing 236. The optical fiber housing 236 houses a portion of the optical fiber 110.

The distal cap 330 includes a connection portion 366 and an angled portion 368, as shown in FIG. 3D. The distal cap 330 may include a metallic material, a silicone material, an elastomeric material, a plastic material, a crystal (e.g., a sapphire), or a ceramic material. The connection portion 366 may be secured to the aspiration tube 238 by threading, welding, or press-fitting. The aspiration tube 238 has an outer diameter D4. The connection portion 366 has an inner diameter D5 which may be the same or substantially the same as the outer diameter D4 of the aspiration tube 238. In some embodiments, the aspiration tube 238 and the distal cap 330 may be a single, monolithic unit.

The distal cap 330 further includes a distal end 352, an optical fiber port 354, a channel 356, and a distal opening 358. The optical fiber port 354 may partially surround the optical fiber 110. For example, the optical fiber port 354 may partially annularly surround the optical fiber 110. The optical fiber port 354 terminates at the distal opening 358. In various embodiments, the optical fiber guide 254 may position the optical fiber 110 within a portion of the channel 256 at the distal opening 258 to allow the optical fiber 110 to deliver laser beams to the surgical site. In some embodiments, a distal end 260 of the optical fiber 110 is coplanar or substantially coplanar with the distal opening 358.

The distal end 352 may include a distal flange 362, as shown in FIG. 3C, extending away from the distal opening 358 below the optical fiber port 354. A distal surface 364 is formed by the distal flange 362. The distal surface 364 provides for improved followability, purchase, and to prevent burrowing of the probe tip 145. In some embodiments, the distal end 360 of the optical fiber 110 is coplanar or substantially coplanar with the distal surface 364.

The channel 356 extends between the aspiration tube 238 and the distal opening 358. The aspiration tube 238 and the channel 356 define an aspiration pathway 339. The channel 356 may be defined by the angled portion 368. The channel 356 within the angled portion 368 is angled away from the aspiration tube 238 towards the optical fiber port 354 and terminates at the distal opening 358.

The angled portion 368 has a cross-sectional area defined by a diameter D6. In other embodiments, the cross-sectional area may be defined by other dimensions, e.g., height and width, depending on the shape of the angled portion 368. The inner diameter D1 of the aspiration tube 238 may be greater than the diameter D6 of the angled portion 368. The distal opening 358 may have a cross-sectional area equal to that of a distal end of the angled portion 368. In such embodiments, the cross-sectional area of the angled portion 368 therefore limits the size of the pieces of tissue and vitreous that are being aspirated through the aspiration pathway 339.

In various embodiments, the optical fiber port 354 may position the optical fiber 110 within a portion of the channel 356 at the distal opening 358, enabling the optical fiber 110 to more efficiently deliver the laser beams to the surgical site. As a result, tissue and vitreous that is ablated and/or dissolved by the optical fiber 110 can be more effectively aspirated through the aspiration pathway 239. Accordingly, by positioning the optical fiber 110 proximate to the aspiration pathway 239, without being disposed within the aspiration tube 238, the likelihood of clogging is decreased.

Additionally, in various embodiments the optical fiber port 354 may position the optical fiber 110 within a portion of the channel 356 to control the size of the tissue and vitreous which can be aspirated through the cross-sectional area of the distal opening 358. Positioning the optical fiber 110 within a portion of the channel 356 decreases the cross-sectional area of the distal opening 358. The cross-sectional area of the distal opening 358, therefore, limits the size of the pieces of tissue and vitreous that are aspirated through the aspiration pathway 339. As a result, tissue and vitreous that is aspirated through the aspiration pathway is smaller than the cross-section area of the distal opening 358, further decreasing the likelihood that large pieces of tissue and vitreous cause clogging within the aspiration pathway 339.

The distal cap 330 may further comprise an inner flange 380 and an anchor 392. The aspiration tube 238 may comprise an anchor opening 290. The anchor 392 may be configured to be disposed within the anchor opening 290. The inner flange 380 abuts a distal end 294 of the aspiration tube 238. The anchor 392 and inner flange 380 position the aspiration tube 238 within the distal cap 330 to allow for the aspiration tube to efficiently aspirate the tissue and vitreous.

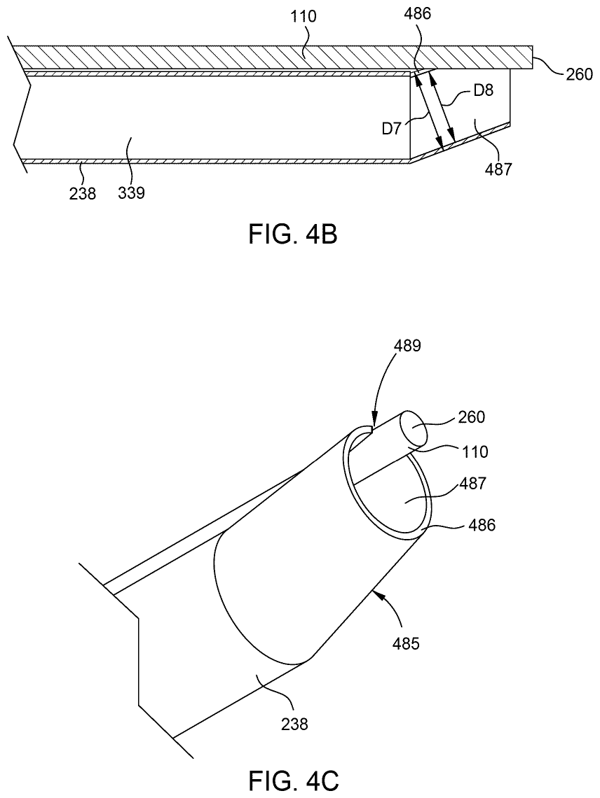

FIG. 4A-4C illustrates a cross-sectional side view of the distal cap 330 with hypotube 485. The hypotube 485 may be disposed at the distal end 294 of the aspiration tube 238 and extend towards the distal opening 358. The hypotube 485 has a sidewall 486 which defines the hypotube channel 487. The hypotube 485 is disposed within the channel 356 of the distal cap 330. The hypotube 485 has an outer diameter D7. The outer diameter D7 of the hypotube 485 may be less than the diameter D6 of the angled portion 368 of the distal cap 330.

The sidewall 486 of the hypotube 485 may be a tapered sidewall having a cross-sectional area defined by the diameter D8, as shown in FIG. 4B. In other embodiments, the cross-sectional area may be defined by other dimensions, e.g., height and width, depending on the shape of the hypotube 485. The diameter D8 decreases as the hypotube 485 approaches the distal opening 358.

In various embodiments, the hypotube 485 may be angled towards the optical fiber port 354 such that the optical fiber port 354 may position the optical fiber 110 within the hypotube channel 487 and a hypotube opening 488, enabling the optical fiber 110 to more efficiently deliver the laser beams to the surgical site. In some embodiments, the optical fiber 110 occupies a portion of the diameter D8 (e.g., cross-sectional area) of the hypotube opening 488, decreasing the cross-sectional area of the hypotube opening 488. The sidewall 486 of the hypotube is configured to have a fiber opening 489 to allow the optical fiber 110 to be disposed within the hypotube opening 488, as shown in FIG. 4C. As a result, tissue and vitreous that is ablated and/or dissolved by the optical fiber 110 can be more effectively aspirated through the aspiration pathway 339. Accordingly, by positioning the optical fiber 110 proximate to the aspiration pathway 339, without being disposed within the aspiration tube 338, the likelihood of clogging is decreased.

In further embodiments, the morphology disclosed above for the distal cap could be formed of two or more components, such as the distal cap and the sleeve. For example, a sleeve may be sized to house the hypotube of FIGS. 4A-4C. In another example embodiment, a distal cap may form a bottom portion of the distal cap 330, while a sleeve may form a top portion of the distal cap. In other additional embodiments, a separate tool may be used to provide irrigation to the patient's eye.

In summation, the embodiments of the present disclosure provide a system in which an optical fiber is disposed external to an aspiration tube. A sleeve or distal cap having a channel forms an aspiration pathway with the aspiration tube. The optical fiber is partially disposed in a distal opening of the sleeve or distal cap in order to guide a laser beam from a laser source to a desired location on a patient's body, such as the lens of the patient's eye. The optical fiber ablates or dissolves tissue and vitreous such that the tissue and vitreous are efficiently aspirated from the patient's eye. The optical fiber being external to the aspiration pathway decreases the likelihood of clogging in the aspiration tube.

The above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description.

What is claimed is:

1. An optical fiber system, comprising:
an aspiration tube;
an optical fiber external to the aspiration tube; and
a sleeve surrounding the optical fiber and the aspiration tube, the sleeve comprising:
a distal portion that defines a channel extending between the aspiration tube and a distal opening of the sleeve, wherein the channel and the aspiration tube define an aspiration pathway, and the optical fiber is partially disposed within the channel.

2. The optical fiber system of claim 1, wherein the channel is angled towards an optical fiber guide and terminates at the distal opening.

3. The optical fiber system of claim 2, wherein the optical fiber guide partially surrounds the optical fiber and terminates at the distal opening.

4. The optical fiber system of claim 3, wherein the distal portion of the sleeve includes a flange extending away from the optical fiber guide.

5. The optical fiber system of claim 4, wherein the flange forms a distal surface.

6. An optical fiber system, comprising:
an aspiration tube;
an optical fiber external to the aspiration tube; and
a distal cap, the distal cap comprising:
a channel extending between the aspiration tube and a distal opening, the channel and the aspiration tube defining an aspiration pathway, the optical fiber being partially disposed within the channel.

7. The optical fiber system of claim 6, wherein the aspiration pathway is further defined by a hypotube positioned at a distal end of the aspiration tube.

8. The optical fiber system of claim 6, wherein an optical fiber port surrounds a distal portion of the optical fiber and terminates at the distal opening of the distal cap.

9. The optical fiber system of claim 8, wherein the channel is angled towards the optical fiber port and terminates at the distal opening.

10. The optical fiber system of claim 9, wherein a distal portion of the distal cap includes a flange extending away from the optical fiber port.

11. An optical fiber system, comprising:
an aspiration tube;
an optical fiber external to the aspiration tube; and
a distal cap, the distal cap comprising:
a hypotube disposed at a distal end of the aspiration tube, the hypotube having a hypotube opening; and
a channel extending between the aspiration tube and the distal opening, the channel and the aspiration tube defining an aspiration pathway, the hypotube being disposed within the channel.

12. The optical fiber system of claim 11, wherein the hypotube and the channel are angled toward an optical fiber port and terminate at the distal opening.

13. The optical fiber system of claim 12, wherein the optical fiber port surrounds a distal portion of the optical fiber and terminates at a distal opening of the distal cap.

14. The optical fiber system of claim 13, wherein a distal portion of the distal cap includes a flange extending away from the optical fiber port.

15. The optical fiber system of claim 14, wherein the flange forms a distal surface.
